(19) United States
(12) Patent Application Publication
(10) Pub. No.: US 2015/0246004 A1
(43) Pub. Date: Sep. 3, 2015

(54) TRANSDERMAL DELIVERY DEVICE AND METHOD

(71) Applicant: QuikMed Pty Ltd, Brisbane (AU)

(72) Inventor: Suzana Hillhouse, Deception Bay (AU)

(73) Assignee: QUIKMED PTY LTD, Brisbane (AU)

(21) Appl. No.: 14/715,306

(22) Filed: May 18, 2015

Related U.S. Application Data
(63) Continuation of application No. 13/121,179, filed on Jun. 17, 2011, now abandoned.

(30) Foreign Application Priority Data
Sep. 26, 2008 (AU) 2008905029

(51) Int. Cl. A61K 9/70; A61K 47/38; A61F 13/40; A61K 31/167

(52) U.S. Cl. CPC A61K 9/7007; A61K 31/167; A61K 47/38; A61M 35/006

(57) ABSTRACT

A transdermal delivery device is provided for delivering at least one active therapeutic compound to rapidly induce a therapeutic effect, the device comprising: a pad for receiving a depot of the at least one therapeutic compound, the pad having a portion of material of high compression resistance; and the device also comprising a backing layer of greater cross-section than the pad. A method for rapidly reducing a therapeutic effect and a transdermal delivery but for delivering at least one therapeutic compound to rapidly induce a therapeutic effect are also provided.

### CROSS REFERENCE TO RELATED APPLICATIONS

This is a continuation of co-pending U.S. patent application Ser. No. 13/121,179, filed Jun. 17, 2011, which is the U.S. National Stage of International Application No. PCT/AU2009/001248, filed Sep. 21, 2009, which was published in English under PCT Article 21(2), which in turn claims the benefit of Australia Application No. 2008905029, filed Sep. 26, 2008. Each of these earlier filed applications is hereby incorporated by reference in its entirety.

### FIELD OF THE INVENTION

The present invention relates to a transdermal delivery device and method for rapid transdermal delivery of active therapeutic compounds, in particular an anaesthetic compound.

### BACKGROUND OF THE INVENTION

Skin is a structurally complex, relatively thick membrane that provides an effective barrier to the entry of substances into the body. To enter the body through the skin, substances must first be able to penetrate the stratum corneum, the outermost layer of the skin, which is generally recognized as being primarily responsible for the skin's barrier properties. The stratum corneum is a thin layer of dense highly keratinised cells approximately 10 to 15 microns thick over most of the body, although thicker in areas such as the soles of the feet and the palms of the hands. Due to the dense packing of these cells, the rate of diffusion of many compounds across the skin is relatively slow especially those substances applied in an ionized form. Once across the stratum corneum, substances must then cross the viable epidermis and diffuse into the papillary dermis where they can enter the capillaries and be absorbed into the systemic circulation, enter lymphatic vessels or diffuse into the dermis and underlying tissue compartments.

Therapeutic delivery devices and systems, including pads, patches and gels, are generally designed to deliver one or more therapeutically active compounds into or through the skin at a predetermined rate over a specific period of time via the area on the skin where the system is applied. Such therapeutic devices and systems may be arranged to induce therapeutic action anywhere between the surface of the skin and the systemic circulation. There are a large number of active compounds which can be applied in this way and their differing chemical, physical and pharmacological characteristics have meant that there are a number of different transdermal delivery devices and systems which are commercially available.

Conventionally, transdermal patches or pads have at least one active compound reservoir, where the therapeutic compound is present in solid, liquid or dispersed molecular form, and an adhesion layer through which the patches or pads connect to the skin. In addition, these patches or pads also usually have a protective backing cover, typically impermeable to the active compound. The patches or pads may also have a membrane in contact with the skin which is capable of regulating the release rate of the compound.

Common uses of such transdermal patches is for sustained release of a therapeutic substance over a long period of time at a generally constant rate. Examples of such patches include nicotine patches and testosterone patches. U.S. Pat. No. 4,784,857 describes the structure of such a patch in which the active agent is placed between the barrier layer and the release controlling layer. The pharmacological active agent is contained in a reservoir comprising a fibrous mat which is capable of absorbing and then releasing the active agent. The patch is designed to be used in for example a 12 hour or 24 hour period and then discarded.

One problem with many conventional transdermal patches is that they are relatively complicated and difficult to manufacture in particular due to the volatility of the pharmacological active substances which consequently may evaporate during production of the patches. This increases the expense of the patches and limits their application to cost effective clinical applications. US RE 37934 E attempts to address this problem by providing a higher concentration depot of the active substance in the reservoir matrix of the patch. US RE 37934 E describes a nicotine patch in which a depot of 140 g nicotine in 100 g of an acrylic resin of dimethylaminoethylmethacrylate and neutral methacrylates is formed in the patch in 102 mg doses. After 24 hours, the nicotine released in vitro from this patch was 56.54 mg per patch.

International patent application no. PCT/US02/34077 describes a transdermal patch commercially available under the name Lidoderm® for treating non-neuropathic pain which provides continuous transdermal delivery of an anesthetic, specifically lidocaine, over extended periods of time to induce analgesia without causing anesthesia. Analgesia is the alleviation of pain whereas anesthesia refers to numbness, complete loss of sensation or paralysis.

Transdermal delivery of anesthetic substances is also provided to induce anesthesia of the nerve endings of the dermis. Application of the anesthetics in this way is typically provided prior to minor procedures such as needle insertion through the skin, biopsies, minor superficial surgeries, the application of laser energy for cutaneous procedures such as the removal of hair and tattoos, for example.

A commercially available product for providing transdermal delivery of an anesthetic to induce the anaethesis is sold under the name EMLA® which comprises a eutectic mixture of local anesthetics lidocaine and prilocaine. EMLA is available as a transdermal patch or as a gel. One significant problem with this product, however, is that it has a very long onset time, typically 45 to 90 minutes or even longer before the dermal anesthetic effect is present to a sufficient degree. Furthermore, deeper dermal anaethesis requires covering the application with an occlusive dressing to enhance penetration, which is inconvenient, messy and an added expense.

Another problem with using EMLA is that prilocaine is known to increase susceptibility to methemoglobinemia, particularly in small children, which impairs the oxygen carrying capacity of haemoglobin. Lidocaine, whilst not susceptible to producing the same side effects, can cause systemic toxicity if used at overly high concentrations in order to increase its transdermal permeability. U.S. Pat. No. 6,299,902 describes a composition to be used as a topical anesthetic with little or no prilocaine so as to avoid the risk of causing methemoglobinemia. The composition of U.S. Pat. No. 6,299,902 has two liquid phases; an aqueous phase and an oil phase, wherein the oil phase has a relatively high concentration of a local anaesthetic agent, preferably lidocaine. Trials of this composition found no significant difference in the latency times between a cream of this composition and EMLA such that “following the application time of 60 minutes, the 6% lidocaine cream and EMLA cream produced comparable anaesthetic effects during the two hour period after removal of the creams”.

A number of other formulations have been reported as suitable compositions to be used in a transdermal delivery device or system to reduce the onset time of anaesthesia compared to that of EMLA:
US2001/00014349 describes a composition comprising “at least one compound (1) modulating the reactivity nerve fibers, at least one compound (2) which is water miscible, solubilises the compound (1) and is volatile, the weight ratio water/volatile compounds being greater than or equal to 0.8, the composition being devoid of any compound, other than water, which does not solubilise the compound (1) and is capable of retarding the evaporation of the volatile compounds present in the composition and devoid of compound which solubilises the compound (1) and is non-volatile”. This composition was found to be efficacious at the end of only 30 minutes.
EP1293203 describes a composition comprising lidocaine with the addition of a volatile carrier/penetration enhancer which is preferably a low carbon alcohol. The minimum time for desensitisation using this composition was reported as 1 hour with the average desensitisation period being reported as 1.5 hours.
US2004/0131665 describes a topical anaesthetic formulation comprising lidocaine, benzylalcohol and isopropyl alcohol which was found to generally produce an anaesthetic effect with 30 minutes.
US2004/0086556 describes a method of enhancing the flux of a local anaesthetic agent through a body surface by administering a basic permeation enhancer to the localised region where a local anaesthetic agent has been administered, the enhancer comprising a pharmaceutically acceptable base and being present in an amount effective to produce a pH in the range of about 8.0 to 13.0. The formulations described in US2004/0086556 were found to provide up to three fold more flux than in the absence of the basic permeation enhancer (NaOH).

Thus, despite many attempts to improve the onset time of anaesthesia over that of EMLA, the onset time remains undesirably long (generally 30 minutes or more). Furthermore, the use of permeation enhancers as described in some of the formulations above can cause undesirable irritation of the skin.

### SUMMARY

According to a first aspect of the present invention there is provided a transdermal delivery device for delivering at least one active therapeutic compound to rapidly induce a therapeutic effect, the device comprising a pad for receiving a depot of the at least one therapeutic compound, the pad having a portion of material of high compression resistance wherein the portion of material of high compression resistance is one layer of the pad, or the pad comprises an outer shell and an inner core and the outer shell is firmer and denser than the inner core; and the device also comprising a backing layer of greater cross-section than the pad.

The device may also comprise an amount of the at least one therapeutic compound deposited within the pad.

The amount of the at least one therapeutic compound may be absorbed within the pad.

The pad and the backing layer may be fixed together.

In another arrangement, the pad and backing layer may be readily separable from another.

The pad may be of any desirable cross-sectional shape such as a square, circle or rectangle.

The pad is generally resistant to compression in a direction through the thickness of the pad such that under compression it does not significantly reduce in thickness. The pad may be not so resistant to lateral compression of the pad.

The pad may have a thickness of 0.5-10 mm.

The pad may comprise a plurality of layers of material. Each layer may have a different composition.

The portion of material of high compression resistance may be one layer of the pad.

The material of high compression resistance may be a cotton material, preferably a compressed cotton material, preferably an unwoven material.

The cotton material may or may not be blended with other fibres such as rayon and synthetic fibres including polyester and nylon for example.

The pad may comprise a layer of plastic foam.

The plastic foam may be polyethylene foam.

The pad may comprise a surface layer formed for example of perforated polyethylene film, nylon net, rayon net or cellulose unwoven cloth for example.

The surface layer may be formed on one face of the pad.

The surface layer may be formed on both faces of the pad.

The surface layer may be formed on the side or sides of the pad.

The pad may comprise an outer shell and an inner core.

The outer shell may be firmer and denser than the inner core.

The outer shell may be formed from perforated polyethylene film, nylon net, rayon net or cellulose unwoven cloth.

The inner core may include the foam layer and the high compression resistant layer.

The pad may be capable of being compressed 0.1-75% of its preloaded thickness.

The pad may have an adsorptivity of 0.001-10 mL/cm³ preferably 0.001 to 1.1 mL/cm³.

The pad may be hydrophilic, or lipophilic or may be a combination of hydrophilic and lipophilic to varying degrees.

The backing layer may be non-elastic or elastic.

The backing layer may have an adhesive surface for adhering to the person's skin.

The backing layer may be impermeable to the at least one active therapeutic compound.

The transdermal delivery device may also comprise an aperture in the backing layer through which an amount of the at least one therapeutic compound may be deposited in the pad.

The transdermal delivery device may also comprise a covering layer for covering the adhesive surface of the backing layer prior to use of the device.

The covering layer is generally removable from contact with the adhesive surface of the backing layer to enable use of the device.

At least one therapeutic compound may be in solid or liquid form.

At least one therapeutic compound may be a powder.

At least one therapeutic compound may be in pure form.

At least one therapeutic compound may be in an unionized form. “Unionized form” is understood to mean that a substantial portion of the therapeutic compound carries no overall charge, either in solution or not.

The at least one therapeutic compound, where in liquid form, may be a solute in a solvent.

The depot may comprise a solution of the at least one therapeutic compound and a solvent.

The at least one therapeutic compound may be at a saturation or near saturation concentration in the solvent.

The at least one therapeutic compound may comprise any one or combination of anaesthetics, corticosteroids, non-steroidal anti-inflammatory agents, analgesics, antifungal agents, nicotine, vasodilators, vasoconstrictors, hypnotically active sedatives, tranquilizers, antihypersensitive agents, diuretics, antibiotics, vitamins, antiepileptic agents, antihistamines, hormones, chemotherapeutic and cytotoxic agents and any other compounds which can be delivered transdermally.

The solvent may comprise any one or combination of water, alcohols, propylene glycol, isopropylmyristate, liquid paraffin, glycerin, acetone, oleic acid, olive oil, essential oils or any other hydrophilic or lipophilic vehicle in which the therapeutic compound (solute) is able to be maintained preferably in an unionized form.

Preferably, the at least one therapeutic compound is any one or more anaesthetic compound.

The anaesthetic compound may be an amine, preferably lidocaine. The anaesthetic compound may be any other agent capable of achieving sufficient anaesthesia via passive skin penetration.

Preferably, the solvent is water.

The lidocaine may have a solubilised concentration in water of 0.001-2%, preferably 0.01-2%, preferably 0.1-2%, preferably 1-2%, preferably 1.5-2.0%, more preferably approximately 2% by weight.

The lidocaine may be at a saturated or near saturated concentration in the solvent.

The transdermal delivery device may also comprise a dye deposited within the pad for indicating the position that the device is placed on the person's skin.

According to a second aspect of the present invention, there is provided a transdermal delivery device for delivering at least one therapeutic compound to rapidly induce a therapeutic effect, the device comprising a pad having a portion of material of high compression resistance wherein the portion of material of high compression resistance is one layer of the pad, or the pad comprises an outer shell and an inner core and the outer shell is firmer and denser than the inner core; a backing layer of greater cross-section than the pad; and an aperture in the backing layer through which an amount of the at least one therapeutic compound may be deposited in the pad.

According to a third aspect of the present invention, there is provided a method for rapidly inducing a therapeutic effect comprising providing a transdermal delivery device comprising a pad having a portion of material of high compression resistance, wherein the portion of material of high compression resistance is one layer of the pad, or the pad comprises an outer shell and an inner core and the outer shell is firmer and denser than the inner core; and a backing layer of greater cross-section than the pad; depositing in the pad an amount of at least one therapeutic compound; and applying the pad to a person's skin to enable the at least one therapeutic compound to rapidly induce the therapeutic effect.

The at least one therapeutic compound may be deposited in the pad during forming of the transdermal delivery device.

The step of depositing may comprise depositing the at least one therapeutic compound in the pad through an aperture in the backing layer.

The step of depositing may comprise contacting the pad with the at least one therapeutic compound.

Contacting the pad with the at least one therapeutic compound may comprise pouring, coating, dipping, swabbing, brushing, or any other suitable contacting step.

The step of depositing may result in the at least one therapeutic compound being absorbed in the pad.

According to a fourth aspect of the present invention, there is provided a method for rapidly inducing a therapeutic effect comprising: providing a transdermal delivery device comprising a pad having a portion of material of high compression resistance, wherein the portion of material of high compression resistance is one layer of the pad, or the pad comprises an outer shell and an inner core and the outer shell is firmer and denser than the inner core; and a backing layer of greater cross-section than the pad, and an amount of at least one therapeutic compound deposited within the pad; and applying the pad to a person's skin to enable the at least one therapeutic compound to rapidly induce the therapeutic effect.

The transdermal delivery device may comprise a device according to the first or second aspect of the present invention.

The therapeutic effect may be sufficiently induced within 15 minutes of application of the pad to the person's skin, preferably within 10 minutes.

The therapeutic effect may be substantially lost within 40 minutes of removal of the pad from the person's skin, preferably within 30 minutes.

The at least one therapeutic compound may be an anaesthetic compound, preferably lidocaine, and the therapeutic effect may be anaesthesia.

The step of applying the pad to the person's skin may comprise applying substantial pressure to the pad towards the person's skin.

The step of applying the pad to the person's skin may comprise fixing the pad to the person's skin.

Fixing the pad to the person's skin may comprise adhering the backing layer to the person's skin, preferably by placing an adhesive surface of the backing layer on the person's skin.

In other arrangement, fixing the pad to the person's skin may comprise wrapping the backing layer around a portion of the person. In this embodiment, the backing layer may be in the form of a bandage or other suitable strip of cloth.

The step of applying the pad to the person's skin may comprise stretching the backing layer of the transdermal delivery device.

According to a fifth aspect of the present invention, there is provided a transdermal delivery kit for delivering at least one therapeutic compound to rapidly induce a therapeutic effect, the kit comprising a transdermal delivery device according to the first or second aspect of the present invention; and an applicator for depositing the at least one therapeutic compound in the pad.

The applicator may be used to deposit the at least one therapeutic compound in the pad through the aperture of the backing layer.

The applicator may comprise a syringe.

The kit may comprise an amount of the at least one therapeutic compound, preferably stored within the applicator prior to use of the kit.

According to a sixth aspect of the present invention, there is provided a transdermal delivery device for delivering at least one active therapeutic compound to rapidly induce a therapeutic effect, the device comprising a pad for receiving a depot of the at least one therapeutic compound, the pad comprising an outer shell and an inner core, the outer shell being firmer and denser than the inner core.

The device may also comprise a backing layer of greater cross-section than the pad.

The outer shell may be formed from a perforated polyethylene film, nylon net, rayon net or cellulose unwoven cloth.

The inner core may comprise a plurality of layers.

The inner core may comprise a layer of plastic foam.

The plastic foam may be polyethylene foam.

The inner core may comprise a layer of compressed cotton material.

The outer shell may comprise a surface layer formed on one face of the pad.

The outer shell may comprise surface layers formed on both faces of the pad.

The outer shell may comprise a surface layer formed on the side or sides of the pad.

### BRIEF DESCRIPTION OF THE DRAWINGS

Embodiments of the present invention will now be described, by way of example only, with reference to the accompanying drawings, in which:

FIG. 1 is cross-sectional schematic view of a transdermal delivery device according to an embodiment of the present invention;

FIGS. 2A and B are exploded views of a pad of the transdermal delivery device according to embodiments of the present invention;

FIG. 3 is cross-sectional schematic view of a transdermal delivery device according to another embodiment of the present invention; and

FIG. 4 is cross-sectional schematic view of a transdermal delivery device according to a further embodiment of the present invention.

FIG. 5 is a schematic representation of one embodiment of inner core 21, showing that inner core 21 may comprise a plurality of layers exemplified by 22a and 22b.

FIG. 6 is a schematic representation of kit 40, which includes device 10 (having pad 11) and applicator 30 (exemplified by a syringe).

### DETAILED DESCRIPTION OF EMBODIMENTS

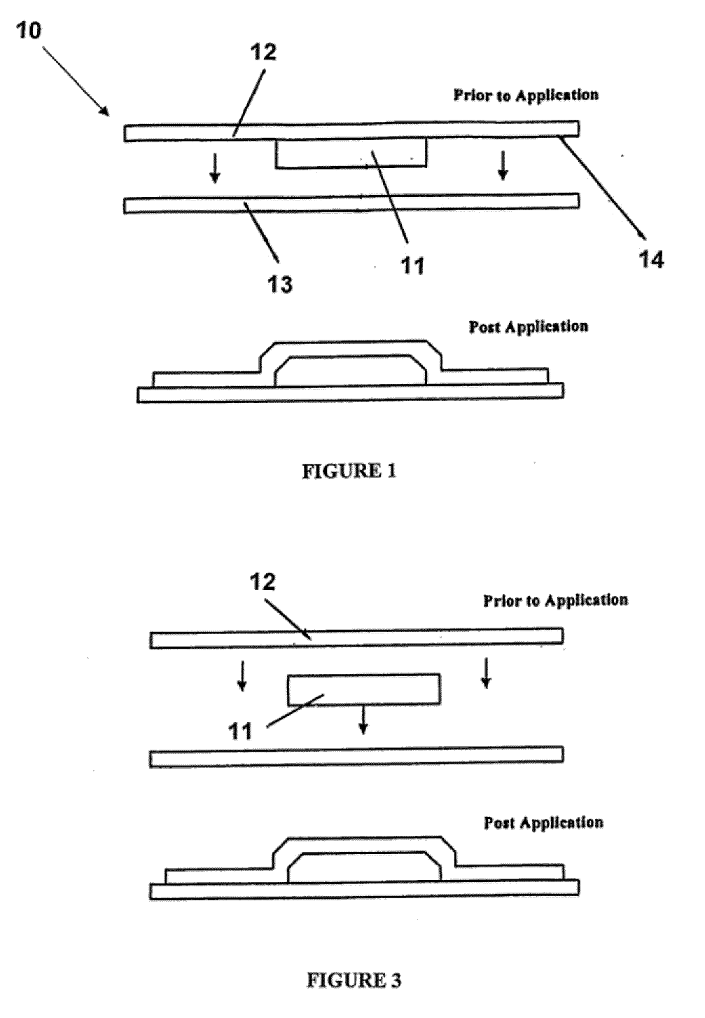

Referring firstly to FIGS. 1 and 3, there is shown a transdermal delivery device 10 for delivering at least one active therapeutic compound to rapidly induce a therapeutic effect. The device 10 comprises a pad 11 for receiving a depot of the at least one therapeutic compound. The pad 11 has a portion of material of high compression resistance. The device 10 also has a backing layer 12 of greater cross-section than the pad 11. The pad 11 is applied to a person's skin 13 to enable transdermal delivery of the at least one therapeutic compound. In the embodiment shown in FIG. 1, the pad 11 and the backing layer 12 are fixed together. Whereas, in the embodiment shown in FIG. 3, the pad and backing layer are readily separable from another.

The pad 11 or at least the portion of the pad of high compression resistance, is generally resistant to compression in a direction through the thickness of the pad 11 such that under compression it does not significantly reduce in thickness, but may be not so resistant to lateral compression. As such, the pad 11 may be commonly referred to as a “pressure pad”. Pressure pads have been conventionally used for aiding in the haemostasis of punctures (ie. blood clotting) by the application of pressure. In addition to having the aforementioned property of not significantly reducing in thickness when under compression, pressure pads are strongly adsorbent of fluids so as to prevent the flow of blood away from the punctures. Surprisingly, it has been found by the inventor of the present invention that such pads may be used in the transdermal delivery device 10 of the present invention.

The pad 11 may be hydrophilic, lipophilic or maybe a combination of hydrophilic and lipophilic, in order to enhance its ability to absorb fluids.

The pad 11 comprises a plurality of layers of material and each layer may have a different composition. The portion of material of high compression resistance typically is one layer of the pad 11. This layer is formed from a compressed unwoven cotton cloth which may or may not be blended with other fibres such as rayon or synthetic fibres including nylon and polyester. The pad 11 may also comprise a “cushioning” layer of polyethylene foam for example. The pad may further comprise a surface layer formed for example from perforated polyethylene film, nylon net, rayon net, or cellulose unwoven cloth.

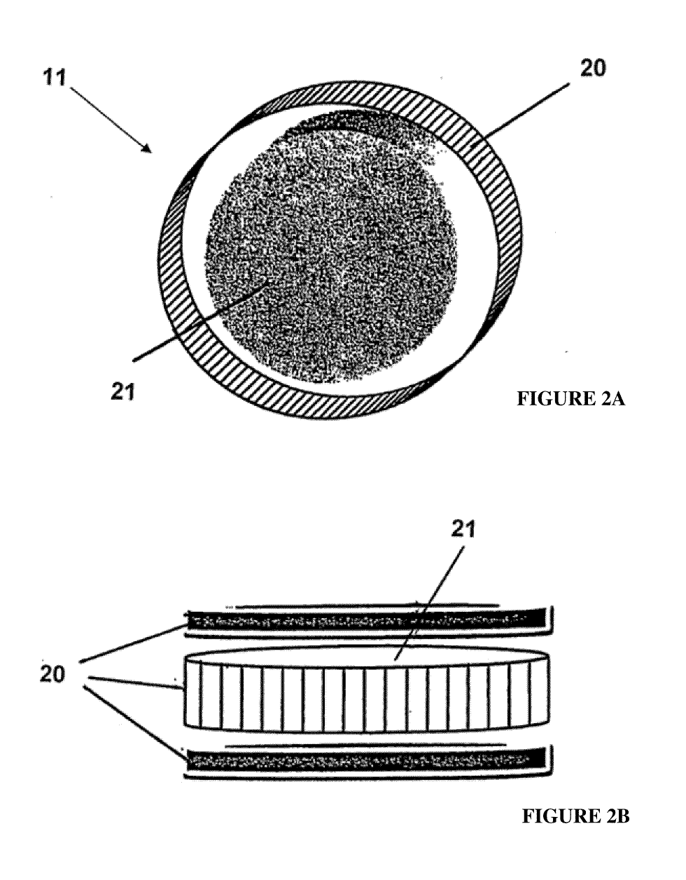

Referring to FIGS. 2A and 2B, in one embodiment, the pad 11 comprises an outer shell 20 and an inner core 21. The outer shell 20 is firmer and denser than the inner core 21. The outer shell 20, as shown in FIGS. 2A and 2B, generally comprises surface layers formed on the faces of the pad and also around the side of the pad. The outer shell 20 is formed preferably from perforated polyethylene film, but may be formed from nylon net, rayon net or cellulose unwoven cloth. The inner core 21 may include a plurality of layers (22a/22b), such as the polyethylene foam layer as well as the compressed unwoven cotton cloth layer. Without wishing to be bound by theory, it is understood that the firmer and denser outer shell 20 reduces undesirable seepage of liquid from the pad 11 under the application of pressure, particularly out of the sides of the pad. The outer shell 20 is also understood to enhance the compression resistance of the pad 11.

The pad 11 is formed such that it generally has the following properties:
a thickness of 0.5-10 mm.
an adsorptivity of 0.001-10 ml/cm³, preferably 0.001-1.1 mL/cm³
is capable of being compressed to 0.1-75% of its preloaded thickness.

The pad 11 may be of any desirable cross-sectional shape such as a square, circle, oval or rectangle.

The backing layer 12 is elastic, which enables it to be stretched as the transdermal delivery device 10 is applied to a person's skin. This substantially increases the pressure applied to the pad 11 towards the person's skin. The backing layer 12 has an adhesive surface 14 for adhering to the person's skin and which also holds the backing layer 12 in its stretched position. The backing layer 12 is also impermeable to the at least one active therapeutic compound so as to avoid any undesirable leaking of the therapeutic compound through the backing layer 12. Although not shown in the Figures, the transdermal delivery device 10 may also comprise a covering layer for covering the adhesive surface 14 of the backing layer 12 prior to use of the device 10. The covering layer is removable from contact with the adhesive surface 14 of the backing layer 12 to enable use of the device 10.

The transdermal delivery device 10 may also have a dye deposited within the pad for indicating the position that the device 10 is placed on the person's skin.

The at least one therapeutic compound may be deposited in the pad 11 during forming of the transdermal delivery device 10. However, in other embodiments this is not the case and the at least one therapeutic compound is deposited in the pad by contacting the pad 11 with the at least one therapeutic compound. Contacting the pad 11 with the at least one therapeutic compound may involve pouring, coating, dipping, swabbing, brushing, or any other suitable contacting step. As a result of contacting the pad 11 with the at least one therapeutic compound, where the at least one therapeutic compound is provided in a liquid, it is generally absorbed in the pad 11.

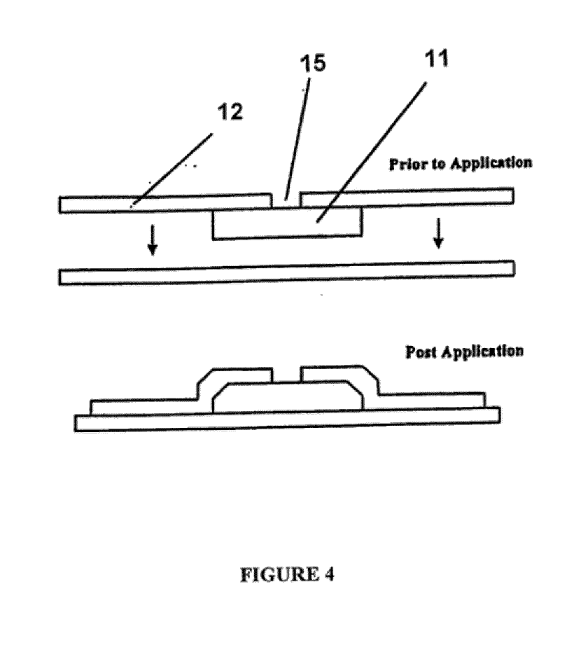

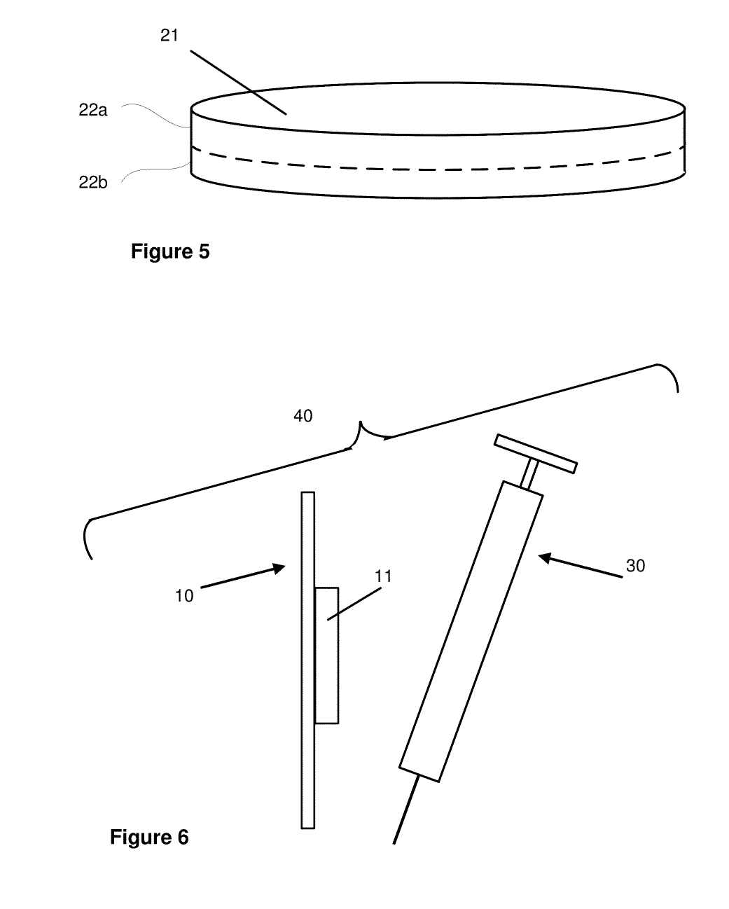

Referring to the embodiment of the device 10 shown in FIG. 4, the device 10 also comprises an aperture 15 in the backing layer 12 through which an amount of the at least one therapeutic compound may be deposited in the pad 11. In this way the at least one compound may be deposited in the pad 11 even after the pad 11 has been applied to the person's skin.

The depot received in the pad 11 generally comprises a solution of the at least one therapeutic compound and a solvent. Although, in other embodiments, the at least one therapeutic compound may be provided in solid or liquid form. Where provided in a solution, the at least one therapeutic compound is preferably in an unionized form at a saturation or near saturation concentration in the solvent.

The at least one therapeutic compound may comprise any one or combination of anaesthetics, corticosteroids, non-steroidal anti-inflammatory agents, analgesics, antifungal agents, nicotine, vasodilators, vasoconstrictors, hypnotically active sedatives, tranquilizers, antihypersensitive agents, diuretics, antibiotics, vitamins, antiepileptic agents, antihistamines, hormones, chemotherapeutic and cytotoxic agents and any other compounds which can be delivered transdermally.

The solvent may comprise any one or combination of water, alcohols, propylene glycol, isopropylmyristate, liquid paraffin, glycerin, acetone, oleic acid, olive oil, essential oils or any other hydrophilic or lipophilic vehicle in which the therapeutic compound(s) (solute) is able to be maintained preferably in an unionized form.

However, in a particular embodiment of the present invention, the at least one therapeutic compound is any one or more anaesthetic compound, preferably an amine, more preferably lidocaine typically at a concentration in a solvent of water of 0.001-4%, preferably approximately 2% by weight (where the lidocaine is at or near a saturated concentration). Where alternative solutions to water are employed the therapeutic compound will need to be adjusted to achieve saturation or near saturation solubility of the therapeutic compound. Using the transdermal delivery device 10 it has been found that the therapeutic effect (anaesthesia) of the lidocaine is sufficiently induced within 15 minutes of application of the pad 11 to the person's skin, preferably within 10 minutes. Furthermore, it has been found that the anaesthetic effect of the lidocaine is substantially lost within 40 minutes of removal of the pad 11 to the person's skin, preferably within 30 minutes. Advantageously, this minimizes unnecessarily prolonged anesthesia of the skin.

Notably, the device 10 may also be provided as part of a kit 40 which also comprises an applicator 30, such as a syringe, for depositing the at least one therapeutic compound in the pad 11. An amount of the at least one therapeutic compound may be stored within the applicator prior to use of the kit.

### EXAMPLES

### Example 1

A transdermal delivery device was produced comprising an adhesive backing layer (3.5 cm diameter circle of non-elastic tape) and a central high compression resistant pad (1.5 cm diameter circle of a fibrous matrix of a sterile cotton band material) attached to the center of the circular backing membrane, 3 mm thickness (uncompressed)). The central pad was loaded with one of the following solutions:

1. 500 μL of a saturated solution of the local anaesthetic lidocaine base (unionized form, approx 2% concentration) in distilled water
2. 500 μL of a 2% lidocaine hydrochloride solution in saline (Xylocaine injection solution).

The device was applied to separate treatment sites for each of the above solutions on the forearm of two female volunteers and the device left in contact with the skin for 10 minutes. After 10 minutes the devices were removed, the surface of the skin blotted with tissue and a 25 g ⅝″ injection needle inserted vertically into the application area on the skin of each site and the depth of penetration of the needle determined at the point when pain was perceived.

In volunteer 1, the needle was able to be inserted to a depth of 5 mm before dermal irritation was perceived following the application of delivery system containing the lidocaine base in water as outlined in 1 above. Insertion of the needle into the site treated with the delivery system containing the active substance depot outlined in 2 above resulted in immediate needle prick pain and no insertion was performed.

In volunteer 2, the needle was also able to be inserted into the dermis before irritation was perceived following the application of delivery system containing the lidocaine base in water as outlined in 1 above. Insertion of the needle into the site treated with the delivery system containing the active substance depot outlined in 2 above again resulted in immediate needle prick pain and no insertion was performed.

These results indicate the achievement of dermal anaesthesia following the short 10 minute application period of the delivery device.

### Example 2

A pad having a diameter of 1.6 cm had 500 μL of 2% lidocaine base (unionized) solution in distilled water deposited therein. The pad was subsequently placed on the inner left forearm of a human volunteer and adhered to skin using a backing layer of surgical tape (3M™ Micropore Surgical Tape). After 15 minutes of application, the pad was removed. A 25 g ⅝″ injection needle was used to prick test the site to around 1 mm depth over two 10 minute intervals; at 10, 20 and 30 minutes after application of the pad. A control site approximately 6 cm from the anaesthetised site was also tested.

The prick testing found that:

after 10 minutes, the area of the skin 1 cm from the pad perimeter was anaesthetized in addition to the area underlying the pad
after 20 minutes, the area of the skin 2.5 cm from the pad was anaesthetized
after 30 minutes, the anaesthetic effect was lost.

### Example 3

A transdermal delivery device is produced comprising a central high compression resistant pad (produced by Nichiban Pty Ltd) and an adhesive backing layer of Opsite cut into a circle of approximately double the diameter of the pad. The device was used in the following trials:

### Trial A

The pad was placed on the adhesive side of the adhesive backing layer and loaded with 0.5 ml of a saturated solution of the local anesthetic lidocaine base (unionised form, approximately 2% concentration) in distilled water. The device was subsequently applied on a volunteer's left deltoid region for ten minutes. A 25 g ⅝″ needle was subsequently inserted in 1 mm increments to a depth of 25 mm (which was the full length of the shaft of the needle). The volunteer did not feel any pain.

### Trial B

The pad was placed on the adhesive side of the adhesive backing layer and loaded with 0.5 ml of a 2% saturated solution of lidocaine base in distilled water. The device was subsequently applied to a volunteer's left deltoid region for 10 minutes and 2 ml of a 1% lidocaine hydrochloride solution in saline (standard Xylocaine injection solution) was infiltrated intradermally on the site where the pad had been applied. No pain was felt by the volunteer during the infiltration of the Xylocaine solution. On a pain scale from 1-10, the volunteer rated it as a 1.

### Trial C

The pad was placed on the adhesive side of the adhesive backing layer and was loaded with 0.5 ml of a 2% lidocaine hydrochloride solution in saline. The device was subsequently applied to a volunteer's right deltoid region for 15 minutes. A 25 g ⅝″ needle was injected in 1 mm increments at the site to a depth of approximately 15 mm before marginal pain was felt by the volunteer.

### Trial D

The pad was placed on the adhesive side of the adhesive backing layer and loaded with 0.5 ml of a 2% lidocaine hydrochloride solution in saline. The device was subsequently applied to a volunteer's right deltoid region for 15 minutes. 2 ml of a 1% lidocaine hydrochloride solution in saline (Xylocaine) was infiltrated intradermally on the site where the pad had been applied. Marginal pain was felt by the volunteer who rated the infiltration of Xylocaine on a pain scale from 1-10 as 3-4.
Trials B and D above compare favourably to the conventional technique for inducing local dermal anesthesia in which 2 ml of Xylocaine (1% lidocaine hydrochloride solution in saline) is injected into a site. Such conventional techniques cause immediate pain upon injection of a needle and subsequently on commencement of infiltration of the Xylocaine solution.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

Variations and modifications can be made in respect of the invention described above and defined in the following statements of claim.

1. A transdermal delivery device for delivering at least one active therapeutic compound to rapidly induce a therapeutic effect, the device comprising:
a pad for receiving a depot of the at least one therapeutic compound, the pad having a portion of material of high compression resistance, wherein the portion of material of high compression resistance is one layer of the pad, or the pad comprises an outer shell and an inner core and the outer shell is firmer and denser than the inner core; and
the device also comprising a backing layer of greater cross-section than the pad.

2. The device according to claim 1 further comprising an amount of the at least one therapeutic compound deposited within the pad.

3. The device according to any one of the preceding claims wherein the pad and the backing layer are fixed together.

4. The device according to claim 1 or 2 wherein the pad and backing layer are readily separable from one another.

5. The device according to any one of the preceding claims wherein the pad has a thickness of 0.5-10 mm.

6. The device according to any one of the preceding claims wherein the pad comprises a plurality of layers of material.

7. The device according to claim 6 wherein each layer of the pad has a different composition.

8. The device according to any one of the preceding claims wherein the material of high compression resistance is a cotton material.

9. The device according to claim 9 wherein the cotton material is blended with fibres selected from rayon, polyester or nylon, or mixtures of two or more of these fibres.

10. The device according to any one of claims 6 to 10 wherein the pad comprises a layer of plastic foam.

11. The device according to any one of the preceding claims wherein the pad comprises a surface layer formed of perforated polyethylene film, nylon net, rayon net or cellulose unwoven cloth.

12. The device according to claim 13 wherein the outer shell is formed from perforated polyethylene film, nylon net, rayon net or cellulose unwoven cloth and the inner core includes a foam layer and a high compression resistant layer.

13. The device according to any one of the preceding claims wherein the pad is capable of being compressed 0.1-75% of its preloaded thickness.

14. The device according to any one of the preceding claims wherein the pad has an adsorptivity of 0.001-10 mL/cm³.

15. The device according to any one of the preceding claims wherein the pad is hydrophilic, or lipophilic or a combination of hydrophilic and lipophilic.

16. The device according to any one of the preceding claims wherein the backing layer has an adhesive surface for adhering to the person's skin.

17. The device according any one of the preceding claims wherein the backing layer is impermeable to the at least one active therapeutic compound.

18. The device according any one of the preceding claims wherein the device further comprises an aperture in the backing layer through which an amount of the at least one therapeutic compound may be deposited in the pad.

19. The device according to any one of the preceding claims wherein the device further comprises a covering layer for covering the adhesive surface of the backing layer prior to use of the device and is removable from contact with the adhesive surface of the backing layer to enable use of the device.

20. The device according to any one of the preceding claims wherein the at least one therapeutic compound is in an unionized form.

21. The device according to any one of the preceding claims wherein the depot comprises a solution of the at least one therapeutic compound and a solvent.

22. The device according claim 23 wherein the at least one therapeutic compound is at a saturation or near saturation concentration in the solvent.

23. The device according to either claim 23 or 24 wherein the solvent comprises any one or combination of water, alcohols, propylene glycol, isopropylmyristate, liquid paraffin, glycerine, acetone, olecic acid, olive oil or essential oils.

24. The device according to any one of the preceding claims wherein the at least one therapeutic compound comprises any one or combination of anaesthetics, corticosteroids, non-steroidal anti-inflammatory agents, analgesics, antifungal agents, nicotine, vasodilators, vasoconstrictors, hypnotically active sedatives, tranquilizers, antihypersensitive agents, diuretics, antibiotics, vitamins, antiepileptic agents, antihistamines, hormones, chemotherapeutic and cytotoxic agents.

25. The device according to any one of the preceding claims wherein the at least one therapeutic compound is any one or more anaesthetic compound.

26. The device according to claim 25 wherein the anaesthetic compound is an amine.

27. The device according to claim 26 wherein the amine is lidocaine.

28. The device according to claim 27 wherein the lidocaine has a concentration in water of 0.001-4%.

29. The device according to claim 27 wherein the lidocaine has a concentration of approximately 2% by weight.

30. The device according to any one of the preceding claims further comprising a dye deposited within the pad for indicating the position that the device is placed on the person's skin.

31. A transdermal delivery device for delivering at least one therapeutic compound to rapidly induce a therapeutic effect, the device comprising:
a pad having a portion of material of high compression resistance, wherein the portion of material of high compression resistance is one layer of the pad, or the pad comprises an outer shell and an inner core and the outer shell is firmer and denser than the inner core;
a backing layer of greater cross-section than the pad; and
an aperture in the backing layer through which an amount of the at least one therapeutic compound may be deposited in the pad.

32. A transdermal delivery device for delivering at least one active therapeutic compound to rapidly induce a therapeutic effect, the device comprising:
a pad for receiving a depot of the at least one therapeutic compound, the pad having a portion of material of high compression resistance, and the pad comprising an outer shell and an inner core, the outer shell being firmer and denser than the inner core.

33. A method for rapidly inducing a therapeutic effect comprising:
providing a transdermal delivery device comprising a pad having a portion of material of high compression resistance, wherein the portion of material of high compression resistance is one layer of the pad, or the pad comprises an outer shell and an inner core and the outer shell is firmer and denser than the inner core; and a backing layer of greater cross-section than the pad;
depositing in the pad an amount of at least one therapeutic compound; and
applying the pad to a person's skin to enable the at least one therapeutic compound to rapidly induce the therapeutic effect.

34. A method according to claim 33 wherein the at least one therapeutic compound is deposited in the pad during forming of the transdermal delivery device.

35. The method according to claim 33 wherein the step of depositing comprises depositing the at least one therapeutic compound in the pad through an aperture in the backing layer.

36. The method according to claim 33 wherein the step of depositing comprises contacting the pad with the at least one therapeutic compound.

37. A method for rapidly inducing a therapeutic effect comprising:
providing a transdermal delivery device comprising a pad having a portion of material of high compression resistance, wherein the portion of material of high compression resistance is one layer of the pad, or the pad comprises an outer shell and an inner core and the outer shell is firmer and denser than the inner core; and a backing layer of greater cross-section than the pad, and an amount of at least one therapeutic compound deposited within the pad; and
applying the pad to a person's skin to enable the at least one therapeutic compound to rapidly induce the therapeutic effect.

38. The method according to claim 37 wherein the transdermal delivery device comprises a device according to any one of claims 1 to 34.

39. The method according to claim 37 or claim 38 wherein the therapeutic effect is sufficiently induced within 15 minutes of application of the pad to the person's skin.

40. The method according to any one of claims 37 to 39 wherein the therapeutic effect is substantially lost within 40 minutes of removal of the pad from the person's skin.

41. The method according to any one of claims 37 to 40 wherein the at least one therapeutic compound is an anaesthetic compound and the therapeutic effect is anaesthesia.

42. The method according to any one of claims 37 to 41 wherein the step of applying the pad to the person's skin comprises applying substantial pressure to the pad towards the person's skin.

43. The method according to any one of claims 37 to 42 wherein the step of applying the pad to the person's skin comprises fixing the pad to the person's skin.

44. The method according to claim 43 wherein fixing the pad to the person's skin comprises adhering the backing layer to the person's skin.

45. The method according to claim 43 wherein fixing the pad to the person's skin comprises wrapping the backing layer around a portion of the person.

46. The method according to any one of claims 37 to 45 wherein the step of applying the pad to the person's skin comprises stretching the backing layer of the transdermal delivery device.

47. A transdermal delivery kit for delivering at least one therapeutic compound to rapidly induce a therapeutic effect, the kit comprising:
a transdermal delivery device as claimed in any one of claims 1 to 34; and
an applicator for depositing the at least one therapeutic compound in the pad.

48. The kit according to claim 47 wherein the applicator comprises a syringe.

49. The kit according to any one of claim 47 or 48 wherein the kit comprises an amount of the at least one therapeutic compound.

50. The kit according to claim 49 wherein the at least one therapeutic compound is stored within the applicator prior to use of the kit.
